(19) United States
(12) Patent Application Publication
(10) Pub. No.: US 2006/0161167 A1
(43) Pub. Date: Jul. 20, 2006

(54) Acetabular instrument alignment guide

(76) Inventors: Reese Myers, Warsaw, IN (US); Brad Parker, Warsaw, IN (US)

Correspondence Address: Todd T. Taylor;Taylor & Aust, P.C., 142 S. Main Street, P.O. Box 560, Avilla, IN, 46710, US

(21) Appl. No.: 11/037,679

(22) Filed: Jan. 18, 2005

(51) Int. Cl. A61F 2/34

(52) U.S. Cl. 606/91

(57) ABSTRACT

An acetabular instrument alignment guide for aligning at least one orthopaedic instrument relative to an acetabulum in a pelvis. The acetabular instrument alignment guide includes an interchangeable head configured for positioning within the acetabulum where the interchangeable head has a primary axis. A drill guide is releasably connected to the interchangeable head. The drill guide includes a drill bore offset from the primary axis, and the drill bore is configured for aligning a drill relative to the pelvis. The acetabular instrument alignment guide further includes at least one reference pin and an instrument guide connected to the at least one reference pin, the instrument guide being both rotatable and translatable relative to the at least one reference pin. The reference pin is configured for placement in at least one hole in the pelvis produced by the drill.

### BACKGROUND OF THE INVENTION

1. Field of the Invention

The present invention relates to an orthopaedic instrument alignment guide, and, more particularly, to an acetabular instrument alignment guide.

2. Description of the Related Art

In total-hip replacement surgery the hip socket or acetabulum and the femoral head are typically badly deteriorated due to arthritis, or some other condition. In elderly patients particularly, this diseased condition dictates the removal of the head (ball) of the femur and its replacement by a polished metal (or other suitable material) ball with a shaft anchored in the intramedullary canal of the femur. To provide a proper bearing surface for the ball, one that will not limit the normal motion of the leg, it is necessary to reform the normal socket, or acetabulum, reaming away the diseased bone and cartilage to make a new structural base to receive an acetabular cup prosthesis (socket) matched to the artificial femoral head. The artificial socket is affixed within the reformed acetabulum by way of a suitable cement. In order to reduce the likelihood of dislocation of the hip after surgery, it is very important to accurately position the cup within the acetabulum. An acetabular cup positioner can be used to position the acetabular cup and to hold the cup in position while the cement is hardening.

Following dislocation of the anatomical femoral head from its associated acetabulum, the acetabulum is prepared to receive the acetabular cup prosthesis by initially reaming the acetabulum until it dimensionally complements the prosthesis. Often it is extremely difficult to judge the amount of tissue and bone to be removed in the reaming operation in order to insure that the prosthesis properly fits within the prepared cavity. Hence there is a real need for instrumentation that assists the orthopedic surgeon in preparing the acetabulum so that it is properly sized to receive the prosthesis.

In shaping the acetabulum to receive the artificial socket, the acetabulum is undercut with an orthopaedic reamer so as to provide a surface against which the cement and artificial socket are seated and thus anchored to the bony structure of the acetabulum. Acetabular reamers are surgical tools, which are used to cut hemispherical cavities in the acetabulum for the insertion of artificial hip joint socket as described above. An acetabular reamer is typically composed of an acetabular reamer cup mounted on a tool driver, which in turn is mounted in the chuck or alignment block of a portable drill or flexible powered shaft. Acetabular reamer cups have an arrangement of precisely shaped cutting surfaces extending outwardly from an essentially hemispherical shell. Acetabular reamer cups are separable from their tool drivers for changing cup size prior to or during surgery, cleaning, and/or sharpening. Acetabular reamers must be capable of producing cavities in the acetabulum with very close tolerances.

In order for the surgeon to produce a reformed acetabulum for an acetabular cup prosthesis which has the required precision, alignment of instruments, such as the positioners and reamers described above, used to machine bone and cartilage and to place components in the acetabulum is required. As these instruments are repetitively placed in the acetabulum to sequentially machine features or to place trials or implants, the orientation of each instrument must be re-verified at each sequence. With each re-verification, there is an opportunity for error, and further, the re-verifications are time consuming. Therefore, the re-verifications tend to decrease the accuracy of the surgical procedures and increase the time required for the surgical procedures thereby increasing the cost of the surgery.

What is needed in the art is an apparatus which eliminates the need for re-verification of orthopaedic instruments in hip replacement surgery.

### SUMMARY OF THE INVENTION

The present invention provides a drill guide which is used to position an associated reference component relative to a pelvis, where the reference component is verified initially and subsequent instruments are positioned relative to the reference component.

The invention comprises, in one form thereof, an acetabular instrument alignment guide for aligning at least one orthopaedic instrument relative to an acetabulum in a pelvis. The acetabular instrument alignment guide includes an interchangeable head configured for positioning within the acetabulum where the interchangeable head has a primary axis. A drill guide is releasably connected to the interchangeable head. The drill guide includes a drill bore offset from the primary axis, and the drill bore is configured for aligning a drill relative to the pelvis. The acetabular instrument alignment guide further includes at least one reference pin and an instrument guide connected to the at least one reference pin, the instrument guide being both rotatable and translatable relative to the at least one reference pin. The at least one reference pin is configured for placement in at least one hole in the pelvis produced by the drill.

An advantage of the present invention is that it eliminates the need for re-verification of orthopaedic instruments in hip replacement surgery.

Another advantage of the present invention is that it saves surgical time in hip replacement surgery.

Yet another advantage of the present invention is that it increases the accuracy of many of the procedures used in hip replacement surgery.

Yet another advantage of the present invention is that it can be used in minimally invasive surgical procedures.

Yet another advantage of the present invention is that it can be used with existing orthopaedic instruments.

Yet another advantage of the present invention is that it allows both translation and rotation degrees of freedom for the alignment of the orthopaedic instruments relative to the acetabulum.

Yet another advantage is that the interchangeable head allows the acetabular instrument alignment guide of the present invention to be used with a variety of different size acetabulums.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of an embodiment of an acetabular instrument alignment guide according to the present invention and placed in an acetabulum;

FIG. 2 is an exploded perspective view of a drill guide assembly of the acetabular instrument alignment guide of FIG. 1;

FIG. 3 is a perspective view of the assembled drill guide assembly of FIG. 2, and placed in an acetabulum, which illustrates how the assembled drill guide assembly is used to drill a reference hole in the pelvis;

FIG. 4 is a perspective view of an alignment jig of the acetabular instrument alignment guide of FIG. 1;

FIG. 5 is a cross-sectional view of the alignment jig of taken along line 5-5 in FIG. 4;

FIG. 6 is a perspective view of the alignment jig of FIG. 4, and placed in an acetabulum, which illustrates how a reference pin of the alignment jig is placed in the reference hole in the pelvis; FIG. 7 is a perspective view of the alignment jig used with an orthopaedic instrument to align the orthopaedic instrument to the acetabulum; and

FIG. 8 is a perspective view of the alignment jig used with an orthopaedic instrument where the alignment block of the alignment jig is rotated relative to the slide arm.

Corresponding reference characters indicate corresponding parts throughout the several views. The exemplification set out herein illustrates one preferred embodiment of the invention, in one form, and such exemplification is not to be construed as limiting the scope of the invention in any manner.

### DETAILED DESCRIPTION OF THE INVENTION

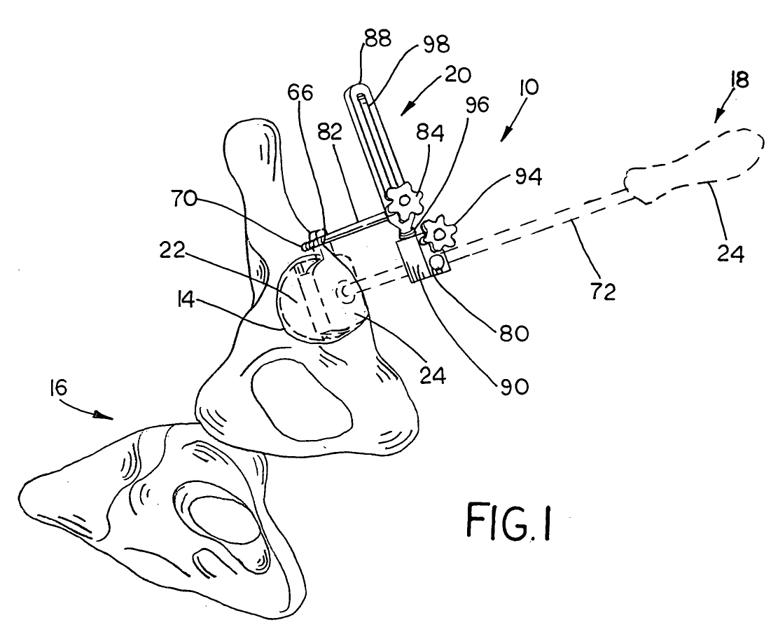

Referring now to the drawings, and more particularly to FIGS. 1 and 7, there is shown an acetabular instrument alignment guide 10 for aligning at least one orthopaedic instrument 12 relative to an acetabulum 14 in a pelvis 16. Acetabular instrument alignment guide 10 generally includes a drill guide assembly 18, configured for positioning within acetabulum 14, and alignment jig 20. In FIG. 1, drill guide assembly 18 is shown in hidden lines because drill guide assembly 18 and alignment jig 20 are not necessarily used conjunctively, as is described below, although they can be used conjunctively.

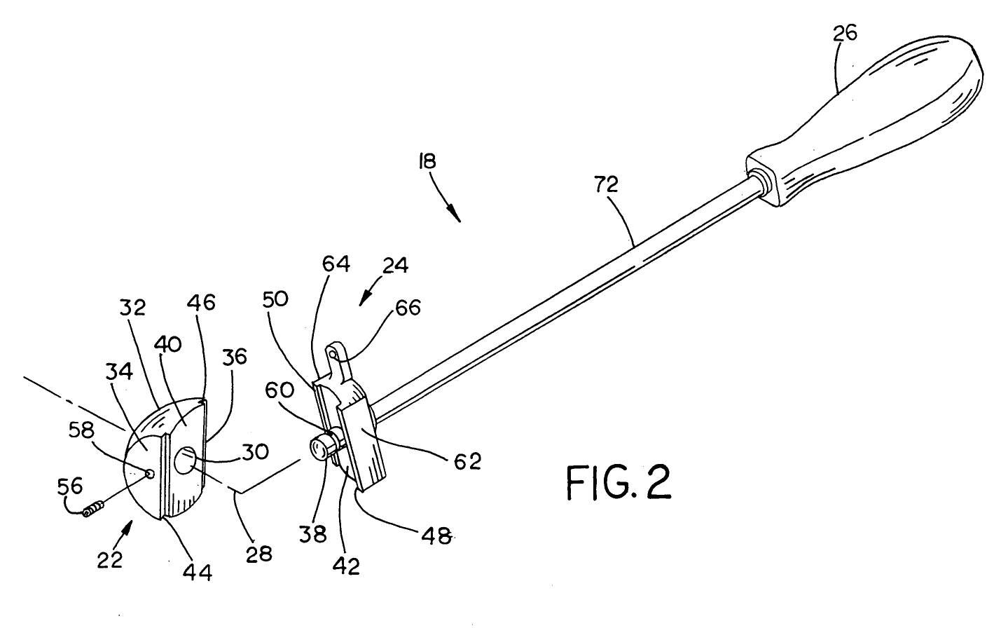

As shown in FIG. 2, Drill guide assembly 18 includes an interchangeable head 22, a drill guide 24 releasably connected to interchangeable head 22 and a handle 26 connected to drill guide 24. Interchangeable head 22 includes a primary axis 28 through shaft bore 30. Shaft bore 30 extends only partially through interchangeable head 22, and particularly, will not typically extend through partially hemispherical surface 32. Head sides 34, 36 reduce the size of a profile presented by interchangeable head 22 to a surgical incision (not shown) in order to facilitate minimally invasive surgery, for example. Interchangeable head 22 can be of a variety of sizes to accommodate different sizes of acetabulum 14.

Shaft end 38 of drill guide 24 is inserted into shaft bore 30. When interchangeable head 22 is fully inserted onto shaft end 38, head base 40 mates with guide base 42, and recessed edges 44, 46 of interchangeable head 22 mate with protruding edges 48, 50, respectively, of drill guide 24 to fix interchangeable head 22 relative to drill guide 24. Interchangeable head 22 is held in this fixed position, relative to drill guide 24, by tightening set screw 56 into threaded hole 58, and onto groove 60 of shaft end 38. In this fixed position, guide sides 62, 64 are typically at least approximately flush with head sides 34, 36, respectively, to reduce the size of a profile presented by drill guide assembly 18 to a surgical incision (not shown) in order to facilitate minimally invasive surgery, for example.

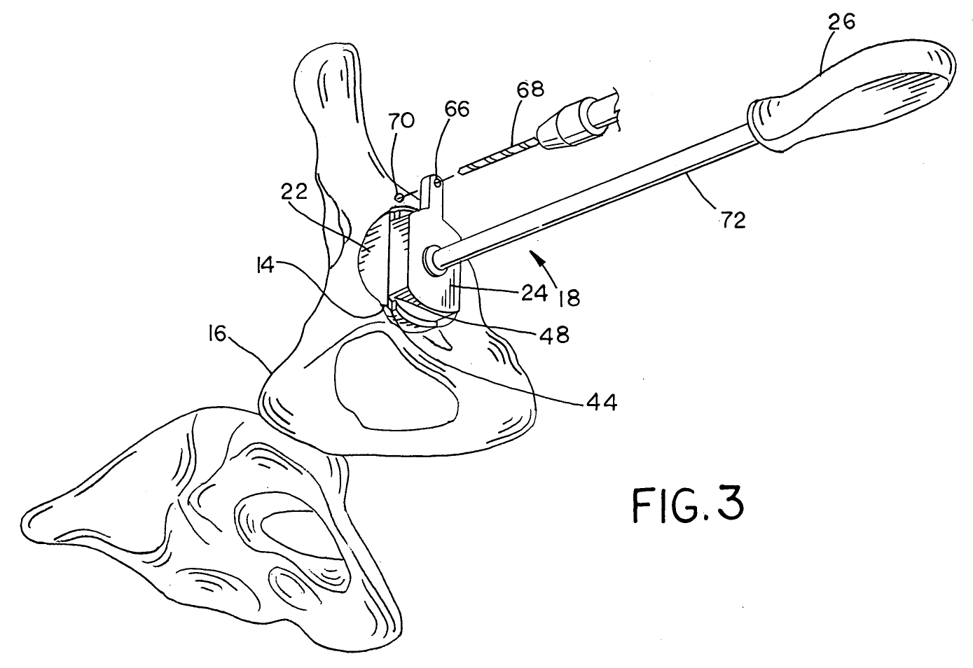

Drill guide 24 includes a drill bore 66 offset from primary axis 28. Drill bore 66 is configured for aligning a drill 68 (FIG. 3) relative to pelvis 16. A surgeon can grasp drill guide assembly 18 by handle 26, insert interchangeable head 22 into acetabulum 14, and appropriately align primary axis 28 relative to acetabulum 14. Primary axis 28 can extend along a longitudinal axis of shaft 72 of drill guide assembly 18. Once drill guide assembly 18 is appropriately aligned according to a surgical procedure, surgical navigation techniques and/or the surgeon's judgement, at least one reference hole 70 is drilled in pelvis 16 using a separate drill 68.

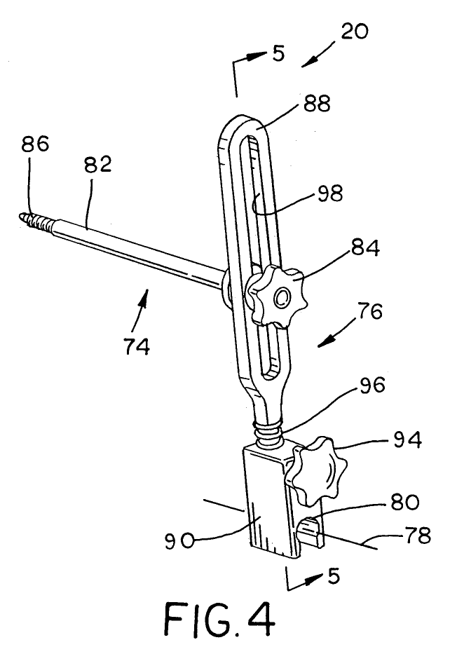

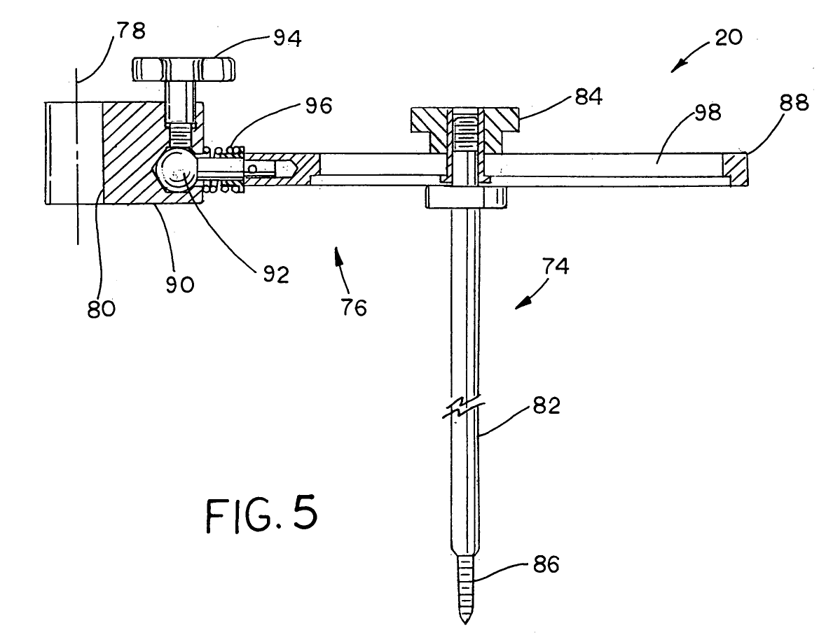

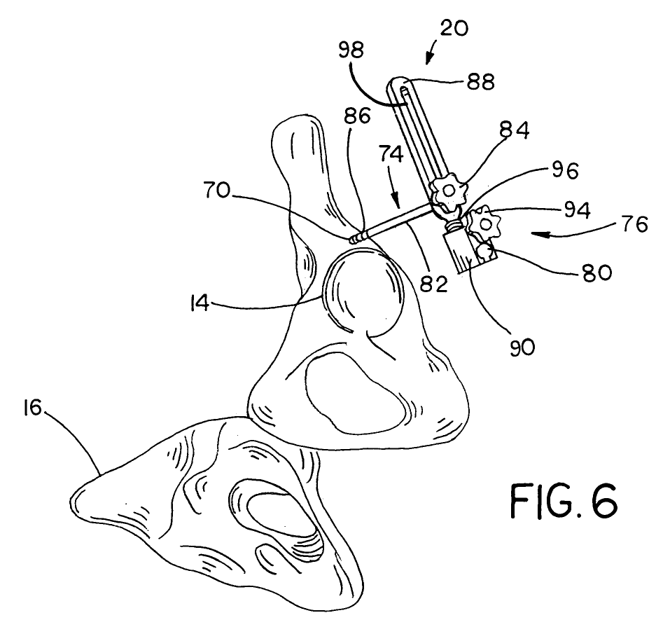

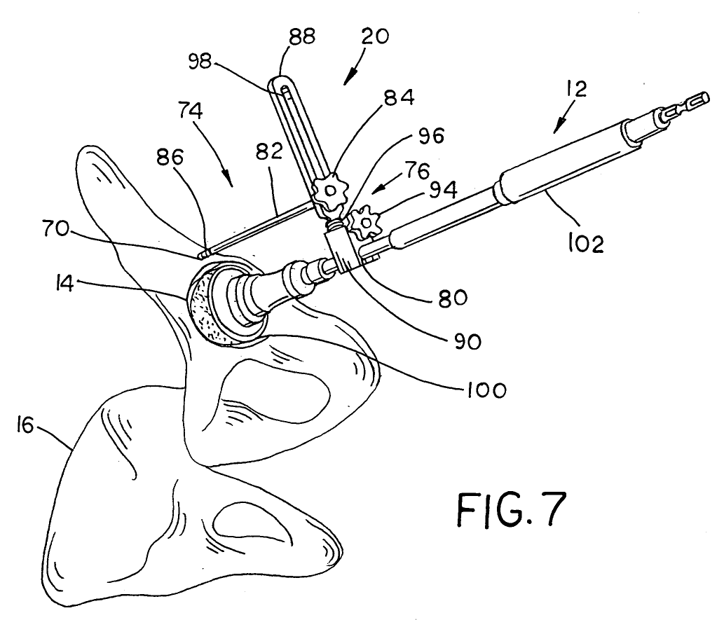

Alignment jig 20 can then be attached to pelvis 16 at reference hole 70. Alignment jig 20 (see particularly FIGS. 4-6) generally includes at least one reference pin 74 and an instrument guide 76 connected to reference pin 74, where instrument guide 76 is alignable to primary axis 28. Although the drawings show a single reference pin 74, more than one reference pin 74 can be used in alignment jig 20 for increased stability. For example, alignment jig 20 can accommodate a two or three point mount utilizing two or three reference pins 74, respectively. In the case of more than two reference pins 74, the tips of reference pins 74 are not necessarily arranged linearly, i.e. the tips can be arranged to define a plane, for example, or another multi-dimensional surface. Typically, there is one reference hole 70 per reference pin 74 located therein. The alignment of instrument guide 76 to primary axis 28 can include the instance where instrument guide axis 78, of instrument bore 80, is at least approximately coincident with primary axis 28, or alternatively, can include instrument guide axis 78 angularly offset and/or translationally offset from primary axis 28 by amounts either determined and/or predetermined by the surgical procedure and/or the surgeon's judgement.

Reference pin 74 includes anchor screw 82 connected to slide lock nut 84. Screw threads 86 allow reference pin 74 to be fixedly placed in reference hole 70. Instrument guide 76 includes a slide arm 88 connected to an alignment block 90 via ball swivel 92 and swivel lock 94. Resilient member 96 is placed between alignment block 90 and slide arm 88. Alignment block 90 defines instrument bore 80 within alignment block 90. Instrument bore 80 is configured to position at least one orthopaedic instrument 12 relative to acetabulum 14. In the embodiment shown, slide arm 88 is positioned in a direction transverse to instrument bore 80. Slide arm 88 includes a longitudinal direction and a slot 98 oriented in the longitudinal direction. Reference pin 74 is releasably positioned within slot 98. As ball swivel 92 is rotatable within alignment block 90, and as instrument guide 76 is both rotatable and translatable relative to reference pin 74 via slot 98, alignment block 90, and more particularly instrument bore 80, is configured to position orthopaedic instrument 12 in alignment with primary axis 28.

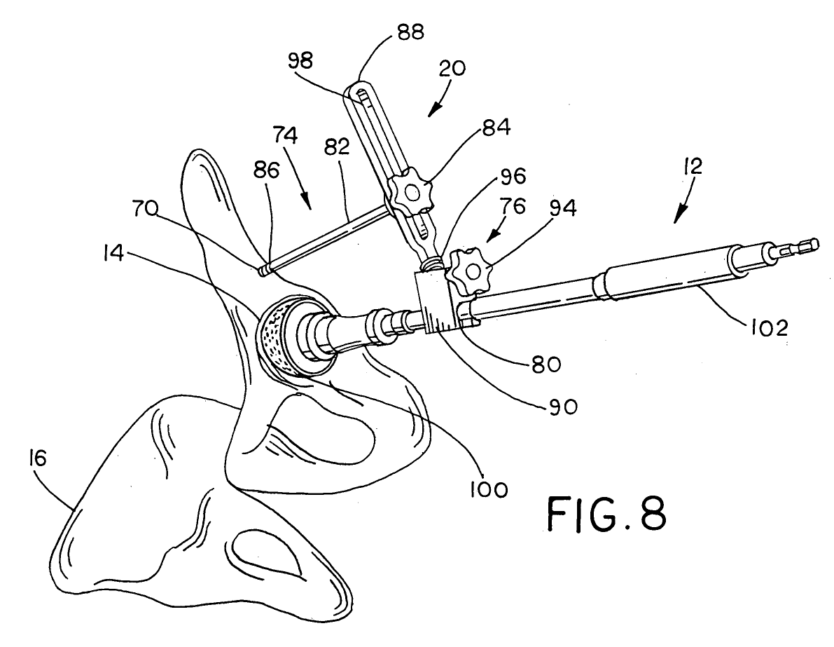

An acetabular instrument kit according to the present invention can include acetabular instrument alignment guide 10 as previously described and at least one orthopaedic instrument 12 which is both alignable to primary axis 28 and configured to be positioned within acetabulum 14. For example, orthopaedic instrument 12 can be an acetabular reamer assembly which includes a reamer 100 and a driver 102 connected to reamer 100, where driver 102 is positionable within alignment block 90. Other embodiments of orthopaedic instrument 12 are contemplated such as a positioner for an acetabular cup prosthesis. As shown in FIG. 8, since alignment block 90 can be rotated relative to slide arm 88, and since instrument guide 76 is both rotatable and translatable relative to reference pin 74, orthopaedic instrument 12 can be aligned to primary axis 28 offset by a predetermined amount. Acetabular instrument alignment guide 10 can be made of stainless steel or other materials suitable to the surgical art.

In use, the present invention discloses a method of aligning orthopaedic instrument 12 relative to acetabulum 14 in pelvis 16, which includes the steps of: positioning an interchangeable head 22 within acetabulum 14, where interchangeable head 22 has a primary axis 28 and is releasably connected to drill guide 24, and drill guide 24 includes a drill bore 66 offset from primary axis 28; drilling at least one reference hole 70 in alignment with drill bore 66 in pelvis 16; affixing at least one reference pin 74 in at least one reference hole 70; aligning an instrument guide 76 to primary axis 28, where instrument guide 76 is connected to at least one reference pin 74; and guiding orthopaedic instrument 12 with instrument guide 76 to perform a surgical procedure. The aligning step can include both rotating and translating instrument guide 76 relative to at least one reference pin 74.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An acetabular instrument alignment guide for aligning at least one orthopaedic instrument relative to an acetabulum in a pelvis, said acetabular instrument alignment guide comprising:
an interchangeable head configured for positioning within the acetabulum, said interchangeable head including a primary axis;
a drill guide releasably connected to said interchangeable head, said drill guide including a drill bore offset from said primary axis.

2. The acetabular instrument alignment guide of claim 1, further including at least one reference pin and an instrument guide connected to said reference pin, said instrument guide being alignable to said primary axis.

3. The acetabular instrument alignment guide of claim 2, wherein said instrument guide is both rotatable and translatable relative to said at least one reference pin.

4. The acetabular instrument alignment guide of claim 2, wherein said instrument guide includes a slide arm connected to both said at least one reference pin and an alignment block which defines an instrument bore within said alignment block, said instrument bore configured to position the at least one orthopaedic instrument relative to the acetabulum.

5. The acetabular instrument alignment guide of claim 4, wherein said slide arm is positioned in a direction transverse to said instrument bore.

6. The acetabular instrument alignment guide of claim 4, wherein said slide arm includes a longitudinal direction and a slot oriented in said longitudinal direction, said at least one reference pin is releasably positioned within said slot.

7. The acetabular instrument alignment guide of claim 4, wherein said instrument bore is configured to position the orthopaedic instrument in alignment with said primary axis.

8. The acetabular instrument alignment guide of claim 1, wherein said drill bore is configured for aligning a drill relative to the pelvis.

9. The acetabular instrument alignment guide of claim 2, wherein said drill bore is configured for aligning a drill relative to the pelvis, said at least one reference pin is configured for placement in at least one hole in the pelvis produced by said drill.

10. The acetabular instrument alignment guide of claim 1, further including a handle connected to said drill guide.

11. An acetabular instrument kit for performing a surgical procedure on an acetabulum in a pelvis, said acetabular instrument kit comprising:
acetabular instrument alignment guide including:
an interchangeable head configured for positioning within the acetabulum, said interchangeable head including a primary axis;
a drill guide releasably connected to said interchangeable positioner head, said
drill guide including a drill bore offset from said primary axis; and
at least one orthopaedic instrument being both alignable to said primary axis and configured to be positioned within the acetabulum.

12. The acetabular instrument kit of claim 11, wherein said acetabular instrument alignment guide further includes at least one reference pin and an instrument guide connected to said reference pin, said instrument guide is alignable to said primary axis.

13. The acetabular instrument kit of claim 12, wherein said instrument guide is both rotatable and translatable relative to said at least one reference pin.

14. The acetabular instrument kit of claim 12, wherein said instrument guide includes a slide arm connected to both said at least one reference pin and an alignment block which defines an instrument bore within said alignment block, said at least one orthopaedic instrument positionable within said instrument bore.

15. The acetabular instrument kit of claim 14, wherein said slide arm is positioned in a direction transverse to said instrument bore.

16. The acetabular instrument kit of claim 14, wherein said slide arm includes a longitudinal direction and a slot oriented in said longitudinal direction, said at least one reference pin is releasably positioned within said slot.

17. The acetabular instrument kit of claim 14, wherein said alignment block is configured to position said at least one orthopaedic instrument in alignment with said primary axis.

18. The acetabular instrument kit of claim 14, wherein at least one said orthopaedic instrument is an acetabular reamer assembly including a reamer and a driver connected to said reamer, said driver is positionable within said alignment block.

19. The acetabular instrument kit of claim 11, wherein said drill bore is configured for aligning a drill relative to the pelvis.

20. The acetabular instrument kit of claim 12, wherein said drill bore is configured for aligning a drill relative to the pelvis, said at least one reference pin is configured for placement in at least one hole in the pelvis produced by said drill.

21. The acetabular instrument kit of claim 11, further including a handle connected to said drill guide

22. A method of aligning an orthopaedic instrument relative to an acetabulum in a pelvis, comprising the steps of:
positioning an interchangeable head within the acetabulum, said interchangeable head including a primary axis, said interchangeable head releasably connected to a drill guide, said drill guide including a drill bore offset from said primary axis;
drilling at least one reference hole in alignment with said drill bore in the pelvis;
affixing at least one reference pin in said at least one reference hole;
aligning an instrument guide to said primary axis, said instrument guide connected to said at least one reference pin; and
guiding the orthopaedic instrument with said instrument guide to perform a surgical procedure.

23. The method of claim 22, wherein said aligning step includes both rotating and translating said instrument guide relative to said at least one reference pin.

24. An acetabular instrument alignment guide for aligning at least one orthopaedic instrument relative to an acetabulum in a pelvis, said acetabular instrument alignment guide comprising: an alignment jig having at least one reference pin and an instrument guide connected to said at least one reference pin, said instrument guide including a slide arm and an alignment block which defines an instrument bore within said alignment block, said slide arm being connected to both said at least one reference pin and said alignment block, said alignment block being configured to position the at least one orthopaedic instrument positionable within said alignment block.
